(19) United States
(12) Patent Application Publication
(10) Pub. No.: US 2011/0300218 A1
(43) Pub. Date: Dec. 8, 2011

(54) NOVEL SOLID STATE FORMS OF RANOLAZINE SALTS

(75) Inventors: Venkata Subbarao Athukuri, Andhra Pradesh (IN); Saidugari Swamy, Andhra Pradesh (IN); Mayur Devjibhai Khunt, Mendarda (IN); Nitin Sharadehandra Pradhan, Maharashtra (IN)

(73) Assignee: ACTAVIS GROUP PTC EHF, Hafnarfjordur (IS)

(21) Appl. No.: 13/063,029

(22) PCT Filed: Sep. 8, 2009
(86) PCT No.: PCT/IB09/06948

§ 371 Date: Aug. 8, 2011

(30) Foreign Application Priority Data
Sep. 9, 2008 (IN) 2185/CHE/2008

(51) Int. Cl. A61K 9/14; A61K 31/495; A61P 9/10; C07D 295/145

(52) U.S. Cl. 424/489; 544/400; 514/252.12

(57) ABSTRACT

Provided herein are solid state forms of ranolazine salts. Also provided is a stable amorphous form of ranolazine hydrochloride having a water content of less than about 0.5% by weight. Further provided are amorphous co-precipitates of ranolazine or a pharmaceutically acceptable salt thereof with povidone. Processes for the preparation of ranolazine forms, pharmaceutical compositions, and methods of treating thereof are also included. The solid state forms of ranolazine salts are useful for preparing ranolazine in high purity.

### CROSS REFERENCE TO RELATED APPLICATION

This application claims the benefit of priority to Indian provisional application No. 2185/CHE/2008, filed on Sep. 9, 2008, which is incorporated herein by reference in its entirety.

### FIELD OF THE DISCLOSURE

The present disclosure relates to novel solid state forms of ranolazine salts, processes for their preparation, pharmaceutical compositions, and method of treating thereof.

### BACKGROUND

Ranolazine, 1-[3-(2-methoxyphenoxy-2-hydroxypropyl]-4-[(2,6-dimethylphenyl)aminocarbonylmethyl]piperazine, is an important antianginal and anti-ischemic agent and useful in the treatment of cardiovascular diseases, including arrhythmias, variant and exercise induced angina and myocardial infarction. Ranolazine is represented by the following structural formula:

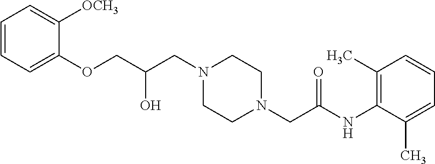

Ranolazine was approved under the brand name RANEXA®, by the U.S. Food and Drug Administration. Ranolazine was first disclosed in U.S. Pat. No. 4,567,264.

U.S. Pat. No. 4,567,264 (herein after referred to as the '264 patent) discloses processes for the preparation of ranolazine or pharmaceutically acceptable salts thereof. While the '264 patent mentions that some of the disclosed compounds can form salts with acids, such as, for example, hydrochloric acid, hydrobromic acid, sulfuric acid, nitric acid, phosphoric acid, acetic acid, propionic acid, glycolic acid, pyruvic acid, oxalic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, only the dihydrochloride salt had been prepared and isolated.

PCT Publication No. WO 2006/008753 A1 (hereinafter referred to as the '753 application) discloses two polymorphic forms (Form A and amorphous form) of ranolazine dihydrochloride and a crystalline form of ranolazine base, processes for the preparation, and characterizes them by powder X-ray diffraction (P-XRD) and Differential Scanning Calorimetry (DSC).

The amorphous form of ranolazine dihydrochloride disclosed in the '753 application is a hydrated form and is not stable, hygroscopic in nature, and not ideal for the preparation of pharmaceutical compositions.

There remains a need for novel solid state forms of ranolazine salts.

### SUMMARY

In one aspect, provided herein are novel solid state forms of a ranolazine salt, wherein the salt is an oxalate salt, a maleate salt, a fumarate salt, a besylate salt, a tosylate salt or a dihydrobromide salt.

In another aspect, ranolazine salts in a crystalline form are provided. In yet another aspect, ranolazine salts in an amorphous form are provided. In still another aspect, the solid state forms of ranolazine salts exist in an anhydrous and/or solvent-free form or as a hydrate and/or a solvate form.

In another aspect, encompassed herein is a process for preparing a solid state form of a ranolazine salt comprising contacting ranolazine free base with a suitable acid in a suitable solvent under suitable conditions to produce a reaction mass, and isolating the solid state form of ranolazine acid addition salt, wherein the acid addition salt of ranolazine is an oxalate salt, a maleate salt, a fumarate salt, a besylate salt, a tosylate salt or a dihydrobromide salt.

In another aspect, provided herein is a method for treating a patient suffering from cardiovascular diseases including arrhythmias, variant and exercise induced angina and myocardial infarction; comprising administering a solid state form of ranolazine salt, or a pharmaceutical composition that comprises the solid state form of ranolazine salt along with pharmaceutically acceptable excipients, wherein the salt of ranolazine is an oxalate salt, a maleate salt, a fumarate salt, a besylate salt, a tosylate salt or a dihydrobromide salt.

In another aspect, provided herein is a pharmaceutical composition that comprises any one of the solid state forms of ranolazine salts disclosed herein, and one or more pharmaceutically acceptable excipients.

In still another aspect, provided herein is a pharmaceutical composition that comprises any one of the solid state forms of ranolazine salts made by the process disclosed herein, and one or more pharmaceutically acceptable excipients.

In still further aspect, encompassed is a process for preparing a pharmaceutical formulation comprising combining any one of the solid state forms of ranolazine salts disclosed herein with one or more pharmaceutically acceptable excipients.

In another aspect, the solid state forms of ranolazine salts disclosed herein for use in the pharmaceutical compositions have a D₉₀ particle size of less than or equal to about 500 microns, specifically less than or equal to about 300 microns, more specifically less than or equal to about 100 microns, still more specifically less than or equal to about 60 microns, and most specifically less than or equal to about 15 microns.

In another aspect, provided herein is a highly stable amorphous form of ranolazine dihydrochloride having a water content of less than about 0.5% by weight based on the total weight of the amorphous ranolazine dihydrochloride (i.e., anhydrous amorphous ranolazine dihydrochloride).

In another aspect, encompassed herein is a process for preparing the highly stable amorphous ranolazine dihydrochloride having a water content of less than about 0.5% by weight based on the total weight of the amorphous ranolazine dihydrochloride.

In one embodiment, the amorphous ranolazine dihydrochloride has a water content of less than about 0.4% by weight, specifically less than about 0.2% by weight, and more specifically less than about 0.1% by weight, and still more specifically is essentially free from water, based on the total weight of the amorphous ranolazine dihydrochloride.

In another aspect, provided herein is a pharmaceutical composition comprising amorphous ranolazine dihydrochloride having a water content of less than about 0.5% by weight, based on the total weight of the amorphous ranolazine dihydrochloride, and one or more pharmaceutically acceptable excipients.

In still another aspect, provided herein is a pharmaceutical composition comprising amorphous ranolazine dihydrochloride having a water content of less than about 0.5% by weight, based on the total weight of the amorphous ranolazine dihydrochloride, made by the process disclosed herein, and one or more pharmaceutically acceptable excipients.

In still further aspect, encompassed herein is a process for preparing a pharmaceutical formulation comprising combining amorphous ranolazine dihydrochloride having a water content of less than about 0.5% by weight, based on the total weight of the amorphous ranolazine dihydrochloride, with one or more pharmaceutically acceptable excipients.

In another aspect, provided herein are amorphous co-precipitates of ranolazine or a pharmaceutically acceptable salt thereof with pharmaceutically acceptable excipients.

In another aspect, encompassed herein is a process for preparing the novel and stable amorphous co-precipitates of ranolazine or a pharmaceutically acceptable salt thereof with pharmaceutically acceptable excipients.

The amorphous co-precipitate of ranolazine or a pharmaceutically acceptable salt thereof obtained by the processes described herein has improved solubility properties and hence also has improved bioavailability.

In another aspect, provided herein are pharmaceutical compositions comprising the amorphous co-precipitates of ranolazine or a pharmaceutically acceptable salt thereof and one or more pharmaceutically acceptable excipients.

In still further aspect, encompassed herein is a process for preparing pharmaceutical formulations comprising combining the amorphous co-precipitates of ranolazine or a pharmaceutically acceptable salt thereof with one or more pharmaceutically acceptable excipients.

In another aspect, the amorphous co-precipitates of ranolazine or a pharmaceutically acceptable salt thereof disclosed herein for use in the pharmaceutical compositions have a D₉₀ particle size of less than or equal to about 500 microns, specifically less than or equal to about 300 microns, more specifically less than or equal to about 200 microns, still more specifically less than or equal to about 100 microns, and most specifically less than or equal to about 15 microns.

### BRIEF DESCRIPTION OF THE DRAWINGS

FIG. 1 is a characteristic powder X-ray diffraction (XRD) pattern of crystalline ranolazine oxalate.

FIG. 2 is a characteristic powder X-ray diffraction (XRD) pattern of crystalline ranolazine maleate.

FIG. 3 is a characteristic powder X-ray diffraction (XRD) pattern of crystalline ranolazine fumarate.

FIG. 4 is a characteristic powder X-ray diffraction (XRD) pattern of crystalline ranolazine besylate.

FIG. 5 is a characteristic powder X-ray diffraction (XRD) pattern of crystalline ranolazine tosylate.

FIG. 6 is a characteristic powder X-ray diffraction (XRD) pattern of amorphous ranolazine dihydrobromide.

FIG. 7 is a characteristic powder X-ray diffraction (XRD) pattern of crystalline ranolazine dihydrobromide.

FIG. 8 is a characteristic powder X-ray diffraction (XRD) pattern of amorphous ranolazine dihydrochloride having a water content of less than 0.5% by weight.

FIG. 9 is a characteristic powder X-ray diffraction (XRD) pattern of an amorphous ranolazine co-precipitate with povidone.

FIG. 10 is a characteristic powder X-ray diffraction (XRD) pattern of an amorphous ranolazine dihydrochloride co-precipitate with povidone.

### DETAILED DESCRIPTION

Solid state forms of ranolazine salts, except dihydrochloride salt, have not been reported, isolated, or characterized in the literature. The present inventors have surprisingly and unexpectedly found that some of the acid addition salts of 1-[3-(2-methoxyphenoxy-2-hydroxypropyl]-4-[(2,6-dimethylphenyl)aminocarbonylmethyl]piperazine, i.e., ranolazine salts, specifically, oxalate, maleate, fumarate, besylate, tosylate and dihydrobromide salts, can be isolated as solid state forms.

It has also been found that the solid state forms of ranolazine salts are useful intermediates in the preparation of ranolazine or a pharmaceutically acceptable salt thereof in high purity. The solid state forms of ranolazine salts have good flow properties are stable at room temperature, enhanced temperature, at relative high humidities, and in aqueous media. The novel solid state forms of ranolazine salts are suitable for formulating ranolazine.

Disclosed herein is the unexpected discovery that ranolazine salts, specifically, oxalate, maleate, fumarate, besylate, tosylate and dihydrobromide salts, can be isolated as solid state forms.

In the formulation of drug compositions, it is important for the active pharmaceutical ingredient to be in a form in which it can be conveniently handled and processed. Convenient handling is important not only from the perspective of obtaining a commercially viable manufacturing process, but also from the perspective of subsequent manufacture of pharmaceutical formulations (e.g., oral dosage forms such as tablets) comprising the active pharmaceutical ingredient.

Chemical stability, solid state stability, and “shelf life” of the active pharmaceutical ingredient are important properties for a pharmaceutically active compound. The active pharmaceutical ingredient, and compositions containing it, should be capable of being effectively stored over appreciable periods of time, without exhibiting a significant change in the physico-chemical characteristics of the active pharmaceutical ingredient, e.g., its chemical composition, density, hygroscopicity and solubility. Thus, in the manufacture of commercially viable and pharmaceutically acceptable drug compositions, it is important, wherever possible, to provide the active pharmaceutical ingredient in a stable form.

New solid state forms of a pharmaceutical agent can further the development of formulations for the treatment of illnesses. For instance, solid forms of a compound are known in the pharmaceutical arts to affect, for example, the solubility, dissolution rate, bioavailability, chemical and physical stability, flowability, fractability, and compressibility of the compound, as well as the safety and efficacy of drug products based on the compound.

The discovery of novel salts in solid state form of pharmaceutically useful compounds provides a new opportunity to improve the performance characteristics of a pharmaceutical product. It also adds value to the material that a formulation scientist can use the same for designing, for example, a pharmaceutical dosage form of a drug with a targeted release profile or other desired characteristic.

According to one aspect, provided herein are novel solid state forms of ranolazine salts, wherein the salt of ranolazine is an oxalate salt, a maleate salt, a fumarate salt, a besylate salt, a tosylate salt or a dihydrobromide salt.

In one embodiment, the solid state forms of ranolazine salts exist in a crystalline form. In another embodiment, the solid state forms of ranolazine salts exist in an amorphous form. In another embodiment, the solid state forms of ranolazine salts exist in an anhydrous and/or solvent-free form or as a hydrate and/or a solvate form. Such solvated or hydrated forms may be present as hemi-, mono-, sesqui-, di- or tri-solvates or hydrates. Solvates and hydrates may be formed as a result of the solvents used during the formation of the ranolazine salts becoming imbedded in the solid lattice structure. Because formation of the solvates and hydrates occurs during the preparation of ranolazine salts, formation of a particular solvated or hydrated form depends greatly on the conditions and method used to prepare the salt. Solvents should be pharmaceutically acceptable.

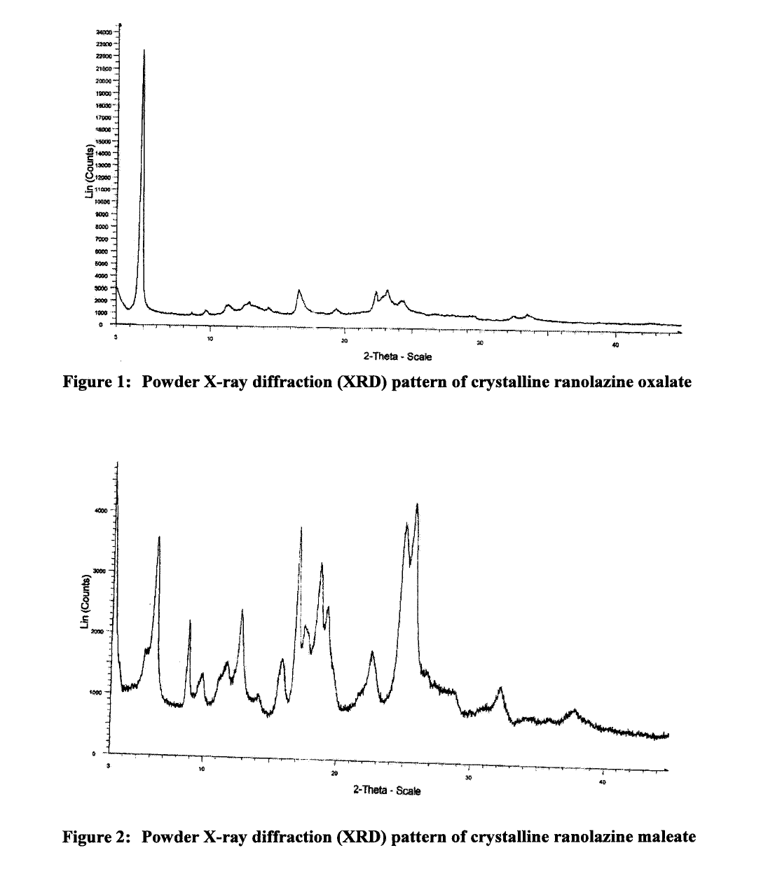

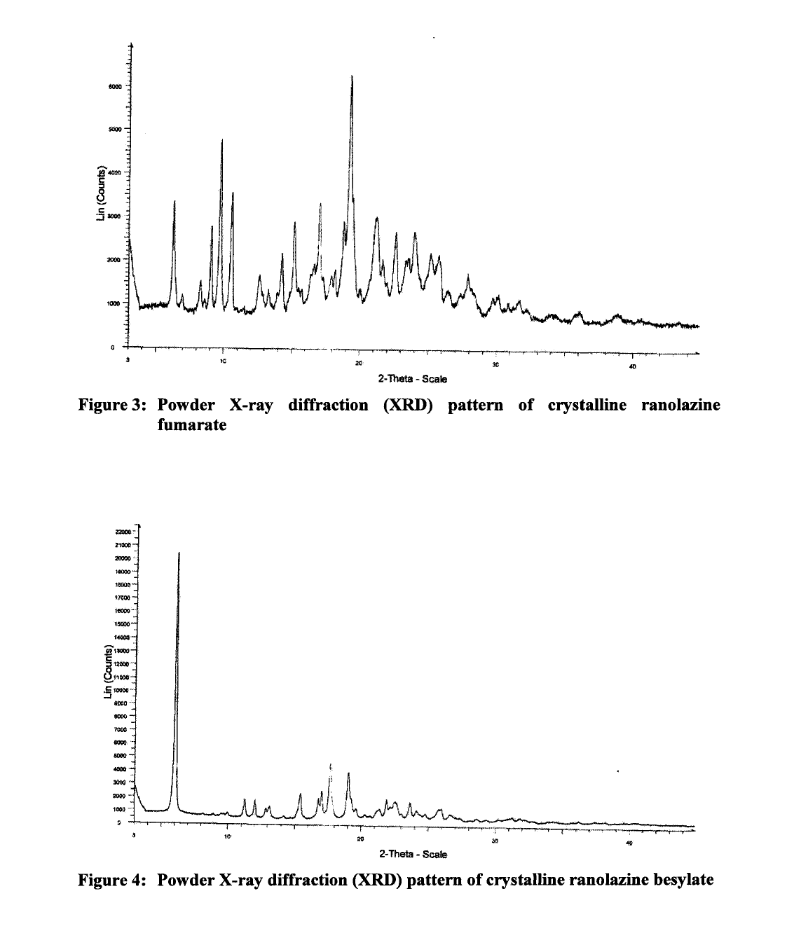

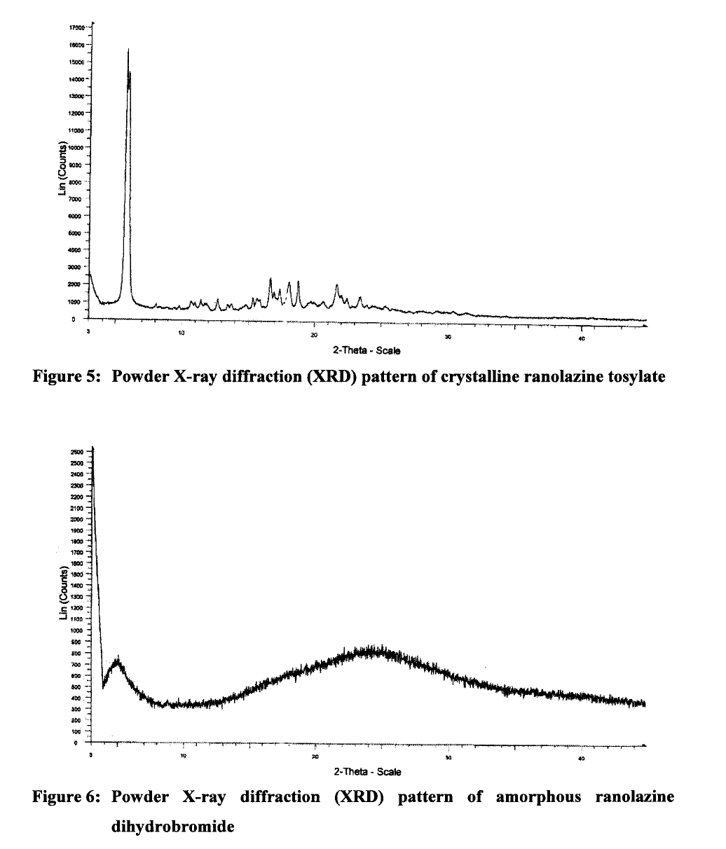

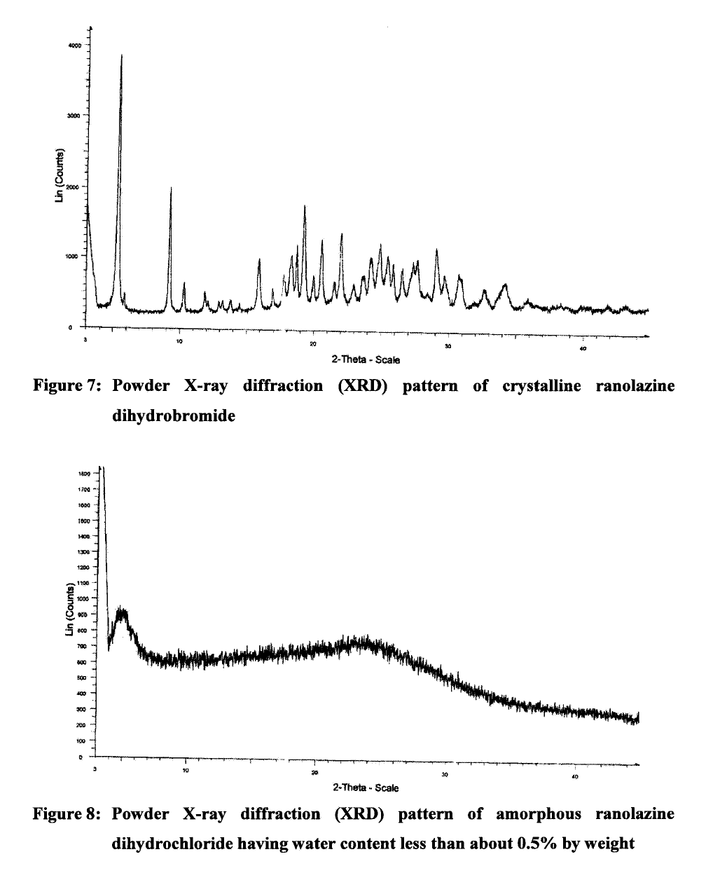

In one embodiment, the solid state forms of ranolazine salts have the following characteristics, wherein:
a) the solid state form of ranolazine oxalate salt is characterized by one or more of the following properties:
   i) a powder X-ray diffraction pattern substantially in accordance with FIG. 1;
   ii) a powder X-ray diffraction pattern having peaks at about 4.79, 16.58, 22.27 and 23.13±0.2 degrees 2-theta; and
   iii) a powder X-ray diffraction pattern having additional peaks at about 9.65, 11.21, 12.60, 12.86, 13.25, 19.36, 22.79, 24.22 and 33.51±0.2 degrees 2-theta;
b) the solid state form of ranolazine maleate salt is characterized by one or more of the following properties:
   i) a powder X-ray diffraction pattern substantially in accordance with FIG. 2;
   ii) a powder X-ray diffraction pattern having peaks at about 6.25, 16.92, 24.82 and 25.54±0.2 degrees 2-theta; and
   iii) a powder X-ray diffraction pattern having additional peaks at about 5.51, 8.78, 9.81, 11.12, 11.69, 12.69, 14.08, 15.82, 17.48, 18.55, 19.14, 19.68, 22.55, 26.68, 27.22, 28.64, 32.19 and 37.81±0.2 degrees 2-theta;
c) the solid state form of ranolazine fumarate salt is characterized by one or more of the following properties:
   i) a powder X-ray diffraction pattern substantially in accordance with FIG. 3;
   ii) a powder X-ray diffraction pattern having peaks at about 9.71, 10.53 and 19.27±0.2 degrees 2-theta; and
   iii) a powder X-ray diffraction pattern having additional peaks at about 6.26, 9.04, 14.25, 15.17, 16.43, 16.62, 17.02, 18.15, 18.81, 19.51, 21.20, 21.66, 22.63, 23.41, 23.56, 24.01, 25.20, 25.81 and 27.91±0.2 degrees 2-theta;
d) the solid state form of ranolazine besylate salt is characterized by one or more of the following properties:
   i) a powder X-ray diffraction pattern substantially in accordance with FIG. 4;
   ii) a powder X-ray diffraction pattern having peaks at about 5.99, 17.64 and 19.04±0.2 degrees 2-theta; and
   iii) a powder X-ray diffraction pattern having additional peaks at about 11.22, 12.01, 15.42, 16.77, 17.02, 21.86 and 23.65±0.2 degrees 2-theta;
e) the solid state form of ranolazine tosylate salt is characterized by one or more of the following properties:
   i) a powder X-ray diffraction pattern substantially in accordance with FIG. 5;
   ii) a powder X-ray diffraction pattern having peaks at about 5.74, 16.65, 18.05 and 18.76±0.2 degrees 2-theta; and
   iii) a powder X-ray diffraction pattern having additional peaks at about 12.68, 15.35, 15.66, 16.94, 17.36, 21.63, 21.96 and 22.40±0.2 degrees 2-theta;
f) the solid state form of ranolazine dihydrobromide salt is characterized by one or more of the following properties:
   i) a powder X-ray diffraction pattern substantially in accordance with FIG. 6; or
   ii) a powder X-ray diffraction pattern substantially in accordance with FIG. 7;
   iii) a powder X-ray diffraction pattern having peaks at about 5.31, 9.18, 15.86, 19.23 and 21.97±0.2 degrees 2-theta; and
   iv) a powder X-ray diffraction pattern having additional peaks at about 10.25, 11.84, 16.92, 17.74, 18.27, 18.70, 19.96, 20.51, 21.49, 23.64, 24.21, 24.88, 25.47, 25.87, 26.57, 27.34, 27.63, 29.05, 29.68, 30.76, 32.64 and 34.20±0.2 degrees 2-theta.

The solid state forms of ranolazine salts are stable, consistently reproducible, and are particularly suitable for bulk preparation and handling. Moreover, the solid state forms of ranolazine salts are useful intermediates in the preparation of ranolazine free base or a pharmaceutically acceptable salt thereof in high purity.

According to another aspect, there is provided a process for the preparation of solid state form of a ranolazine salt, wherein the salt of ranolazine is an oxalate salt, a maleate salt, a fumarate salt, a besylate salt, a tosylate salt or a dihydrobromide salt, comprising:
a) providing a first solution or a suspension of ranolazine free base in a solvent;
b) combining the first solution or suspension with an acid to produce a second solution or suspension containing a ranolazine acid addition salt, wherein the acid is selected from the group consisting of an oxalic acid, a maleic acid, a fumaric acid, a benzenesulfonic acid, a toluenesulfonic acid, and a hydrobromic acid; and
c) isolating and/or recovering the solid state form of ranolazine salt from the second solution or suspension obtained in step-(b).

The solid state form of ranolazine salt obtained by the process disclosed herein is further optionally converted into ranolazine free base or a pharmaceutically acceptable salt thereof by treating the solid state form of ranolazine salt with a base and/or an acid in a solvent.

The process can produce solid state forms of ranolazine salts in substantially pure form.

The term “substantially pure solid state form of ranolazine salt” refers to the solid state form of ranolazine salt having a purity of greater than about 98 wt %, specifically greater than about 99 wt %, more specifically greater than about 99.5 wt %, and still more specifically greater than about 99.9 wt %. The purity is preferably measured by High Performance Liquid Chromatography (HPLC). For example, the purity of solid state form of ranolazine salt obtained by the process disclosed herein can be about 98% to about 99.95%, or about 99% to about 99.99%, as measured by HPLC.

In one embodiment, the process disclosed herein provides stable solid state forms of ranolazine salts. The term “stable solid state form” refers to stability of the solid state form under the standard temperature and humidity conditions of testing of pharmaceutical products, wherein the stability is indicated by preservation of the original polymorphic form.

Exemplary solvents used in step-(a) include, but are not limited to, water, an alcohol, a ketone, a chlorinated hydrocarbon, a hydrocarbon, an ester, a nitrile, an ether, a polar aprotic solvent, and mixtures thereof. The term solvent also includes mixtures of solvents.

In one embodiment, the solvent is selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, n-pentane, n-hexane, n-heptane, cyclohexane, toluene, xylene, tetrahydrofuran, dioxane, diethyl ether, diisopropyl ether, monoglyme, diglyme, N,N-dimethylformamide, N,N-dimethylacetamide, dimethylsulfoxide, and mixtures thereof.

Specifically, the solvent is selected from the group consisting of water, methanol, ethanol, isopropyl alcohol, acetone, methylene chloride, acetonitrile, N,N-dimethylformamide, N,N-dimethylacetamide, dimethylsulfoxide, and mixtures thereof; and more specifically water, methanol, ethanol, isopropyl alcohol, acetone, methylene chloride, and mixtures thereof.

Step-(a) of providing a first solution of ranolazine free base includes dissolving ranolazine free base in the solvent, or obtaining an existing solution from a previous processing step.

In one embodiment, the ranolazine is dissolved in the solvent at a temperature of above about 20° C., specifically at about 25° C. to about 100° C., and more specifically at about 25° C. to about 80° C.

In another embodiment, step-(a) of providing a suspension of ranolazine free base includes suspending ranolazine free base in the solvent while stirring at a temperature of about 0° C. to the reflux temperature of the solvent used. In one embodiment, the suspension is stirred at a temperature of about 20° C. to about 100° C. for at least 30 minutes and more specifically at a temperature of about 25° C. to about 80° C. for about 1 hour to about 10 hours.

In another embodiment, the solution or suspension in step-(a) is prepared by reacting 1-(2-methoxyphenoxy)-2,3-epoxypropane with 4-[(2,6-dimethylphenyl)amino carbonylmethyl]piperazine in a reaction inert solvent under suitable conditions to produce a reaction mass containing ranolazine free base, followed by usual work up such as washings, extractions, evaporations, filtrations, pH adjustments, or a combination thereof. In one embodiment, the work-up includes dissolving, suspending or extracting the resulting ranolazine in the solvent at a temperature of about 0° C. to the reflux temperature of the solvent used, specifically at about 20° C. to about 100° C., and more specifically at about 25° C. to about 80° C.

Alternatively, the solution or suspension in step-(a) is prepared by treating an acid addition salt of ranolazine with a base to produce ranolazine free base followed by extracting, dissolving or suspending the ranolazine in the solvent at a temperature of about 0° C. to the reflux temperature of the solvent used, specifically at about 20° C. to about 100° C., and more specifically at about 25° C. to about 80° C.

In another embodiment, the acid addition salt of ranolazine is derived from a therapeutically acceptable acid such as hydrochloric acid, acetic acid, propionic acid, sulfuric acid, nitric acid, succinic acid, maleic acid, fumaric acid, citric acid, glutaric acid, citraconic acid, glutaconic acid, tartaric acid, malic acid, and ascorbic acid. A specific salt is ranolazine dihydrochloride.

The treatment of an acid addition salt with a base is carried out in a solvent and the selection of solvent is not critical. A wide variety of solvents such as chlorinated solvents, alcohols, ketones, hydrocarbon solvents, esters, ether solvents etc., can be used.

In one embodiment, the base is an organic or inorganic base. Specific organic bases are triethyl amine, trimethylamine and N,N-diisopropylethylamine.

In another embodiment, the base is an inorganic base. Exemplary inorganic bases include, but are not limited to, aqueous ammonia; hydroxides, alkoxides, carbonates and bicarbonates of alkali or alkaline earth metals. Specific inorganic bases are aqueous ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide, and more specifically sodium hydroxide, potassium hydroxide, sodium carbonate and potassium carbonate.

The first solution or suspension obtained in step-(a) is optionally stirred at a temperature of about 25° C. to the reflux temperature of the solvent used for at least 15 minutes, and specifically at a temperature of about 40° C. to the reflux temperature of the solvent used for about 20 minutes to about 8 hours.

As used herein, “reflux temperature” means the temperature at which the solvent or solvent system refluxes or boils at atmospheric pressure.

The acid in step-(b) may be used directly or in the form of a solution containing the acid and a solvent. The solvent used for dissolving the acid is selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, n-pentane, n-hexane, n-heptane, cyclohexane, toluene, xylene, tetrahydrofuran, dioxane, diethyl ether, diisopropyl ether, monoglyme, diglyme, N,N-dimethylformamide, N,N-dimethylacetamide, dimethylsulfoxide, and mixtures thereof.

In one embodiment, the acid used in step-(b) is in a molar ratio of about 1.0 to 5.0 moles, specifically about 2.0 to 2.5 moles, per mole of ranolazine free base.

Combining of the first solution or suspension with acid in step-(b) is done in a suitable order, for example, the first solution or suspension is added to the acid, or alternatively, the acid is added to the first solution or suspension. The addition is, for example, carried out drop wise or in one portion or in more than one portion. The addition is specifically carried out at a temperature of below about 50° C., more specifically at about 15° C. to about 35° C., and most specifically at about 20° C. to about 30° C. under stirring. After completion of the addition process, the resulting mass is stirred at a temperature of about 0° C. to the reflux temperature of the solvent used for at least 10 minutes, specifically at about 15° C. to about 110° C. for about 20 minutes to about 10 hours, and more specifically at a temperature of about 20° C. to about 30° C. for about 30 minutes to about 4 hours to produce a second solution or suspension.

The second solution obtained in step-(b) is optionally subjected to carbon treatment or silica gel treatment. The carbon treatment or silica gel treatment is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing ranolazine acid addition salt by removing charcoal or silica gel. Specifically, the finely powdered carbon is an active carbon. A specific mesh size of silica gel is 40-500 mesh, and more specifically 60-120 mesh.

The isolation of pure solid state form of ranolazine salt in step-(c) is carried out by forcible crystallization, spontaneous crystallization, substantial removal of the solvent from the solution or suspension, or a combination thereof.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution, or a combination thereof.

The term “Anti-solvent” refers to a solvent which when added to an existing solution of a substance reduces the solubility of the substance.

Exemplary anti-solvents include, but are not limited to, a hydrocarbon, an ether, and mixtures thereof. Specifically, the anti-solvent is selected from the group consisting of n-pentane, n-hexane, n-heptane, cyclohexane, toluene, xylene, tetrahydrofuran, dioxane, diethyl ether, diisopropyl ether, monoglyme, diglyme, and mixtures thereof; and most specifically diethyl ether, diisopropyl ether, and mixtures thereof.

In one embodiment, the crystallization is carried out by cooling the solution while stirring at a temperature of below 30° C. for at least 10 minutes, specifically at about 0° C. to about 25° C. for about 30 minutes to about 20 hours.

The term “substantially removing” the solvent refers to at least 80%, specifically grater than about 85%, more specifically grater than about 90%, still more specifically grater than about 99%, and most specifically essentially complete (100%), removal of the solvent from the second solution or suspension.

Removal of solvent is accomplished, for example, by substantially complete evaporation of the solvent, concentrating the solution or distillation of solvent, under inert atmosphere to obtain solid state form of ranolazine salt.

In one embodiment, the solvent is removed by evaporation. Evaporation can be achieved at sub-zero temperatures by lyophilisation or freeze-drying techniques. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying to obtain a dry amorphous powder.

The distillation process can be performed at atmospheric pressure or reduced pressure. Specifically, the solvent is removed at a pressure of about 760 mm Hg or less, more specifically at about 400 mm Hg or less, still more specifically at about 80 mm Hg or less, and most specifically from about 30 to about 80 mm Hg.

Solvents can also be removed by spray-drying, in which a solution of ranolazine salt is sprayed into the spray drier at the flow rate of 10 to 300 ml/hr, specifically 40 to 200 ml/hr. The air inlet temperature to the spray drier used is about 30° C. to about 150° C., specifically about 65° C. to about 110° C. and the outlet air temperature used is about 30° C. to about 90° C.

Another suitable method is vertical agitated thin-film drying (or evaporation). Agitated thin film evaporation technology involves separating the volatile component using indirect heat transfer coupled with mechanical agitation of the flowing film under controlled conditions. In vertical agitated thin-film drying (or evaporation) (ATFD-V), the starting solution is fed from the top into a cylindrical space between a centered rotary agitator and an outside heating jacket. The rotor rotation agitates the downside-flowing solution while the heating jacket heats it.

The recovering in step-(c) is carried out by methods such as filtration, filtration under vacuum, decantation, centrifugation, or a combination thereof. In one embodiment, the solid state form of ranolazine salt is recovered by filtration employing a filtration media of, for example, a silica gel or celite.

The substantially pure solid state form of ranolazine salt obtained by above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 80° C. The drying can be carried out for any desired time period that achieves the desired result, such as about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

The purity of the solid state form of ranolazine salt obtained by the process disclosed herein is greater than about 98%, specifically greater than about 99%, more specifically greater than about 99.9%, and most specifically greater than about 99.95% as measured by HPLC. For example, the purity of the solid state form of ranolazine salt can be about 99% to about 99.95%, or about 99.5% to about 99.99%.

Ranolazine and its dihydrochloride salt can be prepared in high purity by using the substantially pure solid state forms of ranolazine salts obtained according to the process disclosed herein.

According to another aspect, the solid state form of ranolazine dihydrobromide salt disclosed herein is an amorphous ranolazine dihydrobromide salt characterized by a powder X-ray diffraction pattern substantially in accordance with FIG. 6.

According to another aspect, there is provided a process for the preparation of amorphous form of ranolazine dihydrobromide, comprising:
a) providing a solution of ranolazine free base in an alcohol solvent in an amount of less than about 3.5 ml per gram of ranolazine free base;
b) combining the solution obtained in step-(a) with hydrobromic acid; and
c) isolating amorphous form of ranolazine dihydrobromide by adding an anti-solvent.

The process can produce amorphous ranolazine dihydrobromide salt in substantially pure form.

Exemplary alcohol solvents include, but are not limited to, C₁ to C₆ straight or branched chain alcohol solvents such as methanol, ethanol, isopropyl alcohol, butanol, amyl alcohol, hexanol, and mixtures thereof. Specific alcohol solvents are methanol, ethanol, isopropyl alcohol, and mixtures thereof, and a most specific alcohol solvent is methanol.

In one embodiment, the alcohol solvent is used in an amount of about 2 ml to about 3 ml, specifically about 2.2 ml to about 2.8 ml, per gram of ranolazine free base.

In another embodiment, the hydrobromic acid used may be in the form of concentrated hydrobromic acid or aqueous hydrobromic acid, and more specifically in the form of aqueous hydrobromic acid.

Combining of the solution with hydrobromic acid in step-(b) is done in a suitable order as described above.

In one embodiment, the addition of anti-solvent in step-(c) is carried out at a temperature of about 10° C. to about 50° C., specifically at a temperature of about 20° C. to about 40° C. while stirring for at least 10 minutes, and more specifically at about 20° C. to about 30° C. for about 30 minutes to about 4 hours.

The anti-solvent used in step-(c) is selected from the group as described above. Specifically, the anti-solvent is selected from the group consisting of n-pentane, n-hexane, n-heptane, cyclohexane, toluene, xylene, tetrahydrofuran, dioxane, diethyl ether, diisopropyl ether, monoglyme, diglyme, and mixtures thereof; and most specifically diethyl ether, diisopropyl ether, and mixtures thereof.

The amorphous ranolazine dihydrobromide obtained in step-(c) is recovered and further dried by the methods as described above.

According to another aspect, there is provided a process for the preparation of amorphous form of ranolazine dihydrobromide, comprising:
a) providing a first solution of ranolazine free base in a solvent;
b) combining the first solution obtained in step-(a) with hydrobromic acid to produce a second solution; and
c) substantially removing the solvent from the second solution to produce amorphous form of ranolazine dihydrobromide.

The process can produce amorphous ranolazine dihydrobromide salt in substantially pure form.

Exemplary solvents used in step-(a) include, but are not limited to, water, an alcohol, a ketone, a chlorinated hydrocarbon, a nitrile, and mixtures thereof. The term solvent also includes mixtures of solvents.

Specifically, the solvent is selected from the group consisting of water, methanol, ethanol, isopropyl alcohol, acetone, methylene chloride, acetonitrile, and mixtures thereof; and more specifically methanol, ethanol, isopropyl alcohol, acetone, methylene chloride, and mixtures thereof.

In one embodiment, the hydrobromic acid used may be in the form of concentrated hydrobromic acid or aqueous hydrobromic acid or in the form of hydrobromic acid dissolved in an organic solvent selected from the group consisting of an alcohol, a ketone, a chlorinated hydrocarbon, a nitrile, and mixtures thereof.

Combining of the first solution with hydrobromic acid in step-(b) is done in a suitable order as described above.

The removal of the solvent from the second solution in step-(c) is carried out by the methods as described above.

The amorphous ranolazine dihydrobromide obtained in step-(c) is recovered and further dried by the methods as described above.

The purity of the amorphous ranolazine dihydrobromide obtained by the processes disclosed herein is of greater than about 99%, specifically greater than about 99.5%, and more specifically greater than about 99.95% as measured by HPLC.

According to another aspect, the solid state form of ranolazine dihydrobromide salt disclosed herein is a crystalline ranolazine dihydrobromide salt characterized by a powder X-ray diffraction pattern substantially in accordance with FIG. 7.

According to another aspect, there is provided a process for the preparation of crystalline ranolazine dihydrobromide salt, comprising:
a) providing a solution of ranolazine free base in an alcohol solvent in an amount of at least about 4.5 ml per gram of ranolazine free base;
b) combining the solution obtained in step-(a) with hydrobromic acid; and
c) isolating crystalline form of ranolazine dihydrobromide by adding an anti-solvent.

The process can produce crystalline ranolazine dihydrobromide salt in substantially pure form.

The alcoholic solvent used in step-(a) is selected from the group as described above. Specific alcohol solvents are methanol, ethanol, isopropyl alcohol, and mixtures thereof, and a most specific alcohol solvent is methanol.

In one embodiment, the alcohol solvent is used in an amount of about 5 ml to about 50 ml, specifically about 5 ml to about 10 ml, per gram of ranolazine free base.

In another embodiment, the hydrobromic acid used is in the form of concentrated hydrobromic acid or aqueous hydrobromic acid, and more specifically in the form of aqueous hydrobromic acid.

Combining of the solution with hydrobromic acid in step-(b) is done in a suitable order as described above.

In one embodiment, the addition of anti-solvent in step-(c) is carried out at a temperature of about 10° C. to about 50° C., specifically at a temperature of about 20° C. to about 40° C. while stirring for at least 10 minutes, and more specifically at about 20° C. to about 30° C. for about 30 minutes to about 4 hours.

The anti-solvent used in step-(c) is selected from the group as described above. Specifically, the anti-solvent is selected from the group consisting of n-pentane, n-hexane, n-heptane, cyclohexane, toluene, xylene, tetrahydrofuran, dioxane, diethyl ether, diisopropyl ether, monoglyme, diglyme, and mixtures thereof; and most specifically diethyl ether, diisopropyl ether, and mixtures thereof.

The crystalline ranolazine dihydrobromide obtained in step-(c) is recovered and further dried by the methods as described above.

It has been unexpectedly found that a uniformly amorphous form of ranolazine dihydrochloride having a water content of less than 0.5% by weight, based on the total weight of the amorphous ranolazine dihydrochloride, can be obtained in a simple and reproducible process.

Extensive laboratory and full-scale research has resulted in a new and inventive process for producing a highly stable and substantially pure amorphous form of ranolazine dihydrochloride having a water content of less than 0.5% by weight, based on the total weight of the amorphous ranolazine dihydrochloride. The amorphous ranolazine dihydrochloride having a water content of less than 0.5% by weight, based on the total weight of the amorphous ranolazine dihydrochloride, can be utilized to prepare stable pharmaceutical dosage forms having good dissolution properties.

According to one aspect, there is provided a stable and substantially pure amorphous form of ranolazine dihydrochloride having a water content of less than 0.5% by weight, based on the total weight of the amorphous ranolazine dihydrochloride (anhydrous amorphous ranolazine dihydrochloride).

The amorphous form of ranolazine dihydrochloride having a water content of less than 0.5% by weight, based on the total weight of the amorphous ranolazine dihydrochloride, is characterized by a powder XRD pattern substantially in accordance with FIG. 8. The X-ray powder diffraction pattern shows no peaks, thus demonstrating the amorphous nature of the product.

According to another aspect, there is provided a process for the preparation of amorphous ranolazine dihydrochloride having a water content of less than 0.5% by weight, based on the total weight of the amorphous ranolazine dihydrochloride, comprising:
a) providing a solution of ranolazine dihydrochloride in a solvent medium comprising methylene chloride and an alcohol solvent;
b) optionally, filtering the solvent solution to remove any extraneous matter; and
c) substantially removing the solvent from the solution to afford anhydrous amorphous form of ranolazine dihydrochloride.

The process can produce anhydrous amorphous ranolazine dihydrochloride salt in substantially pure form.

The term “substantially pure amorphous ranolazine dihydrochloride” refers to the amorphous ranolazine dihydrochloride having purity greater than about 99%, specifically greater than about 99.5%, more specifically greater than about 99.8% and still more specifically greater than about 99.9% (measured by HPLC).

In a preferred embodiment, the amorphous ranolazine dihydrochloride has a water content of less than about 0.4% by weight, specifically less than about 0.2% by weight, and more specifically less than about 0.1% by weight, and still more specifically is essentially free from water, based on the total weight of the amorphous ranolazine dihydrochloride.

The amorphous ranolazine dihydrochloride having a water content of less than 0.5% by weight obtained by the process disclosed herein is stable, consistently reproducible and has good flow properties, and which is particularly suitable for bulk preparation and handling, and so, the amorphous ranolazine dihydrochloride having a water content of less than 0.5% by weight obtained by the process disclosed herein is suitable for formulating ranolazine dihydrochloride.

The alcoholic solvent used in step-(a) is selected from the group as described above. Specific alcohol solvents are methanol, ethanol, isopropyl alcohol, and mixtures thereof, and a most specific alcohol solvent is methanol.

The solution obtained in step-(a) is optionally subjected to carbon treatment or silica gel treatment as per the methods described above.

The removal of the solvent from the solution in step-(c) is carried out by the methods as described above.

The amorphous ranolazine dihydrochloride having a water content of less than 0.5% by weight obtained in step-(c) is recovered and further dried by the methods as described above.

According to another aspect, there is provided an amorphous co-precipitate comprising ranolazine or a pharmaceutically acceptable salt thereof and povidone (polyvinylpyrrolidone), having improved physiochemical characteristics that assist in the effective bioavailability of ranolazine or a pharmaceutically acceptable salt thereof.

In one embodiment, the povidone may be chosen from one or more of the grades such as PVP K-15, K-25, K-30, K29/32, K-60, K-90, and mixtures thereof.

Exemplary pharmaceutically acceptable salts of ranolazine include, but are not limited to, dihydrochloride, dihydrobromide, oxalate, maleate, fumarate, besylate, tosylate and tartrate. A specific pharmaceutically acceptable salt of ranolazine is dihydrochloride salt.

According to another aspect, there are provided pharmaceutical compositions comprising amorphous co-precipitate of ranolazine or a pharmaceutically acceptable salt with povidone, and one or more pharmaceutically acceptable excipients.

The amorphous co-precipitates of ranolazine or a pharmaceutically acceptable salt thereof with povidone obtained by the processes disclosed herein are characterized by any of their powder X-ray diffraction (XRD) pattern, infrared absorption (IR) spectrum, and SEM images of the morphological analysis.

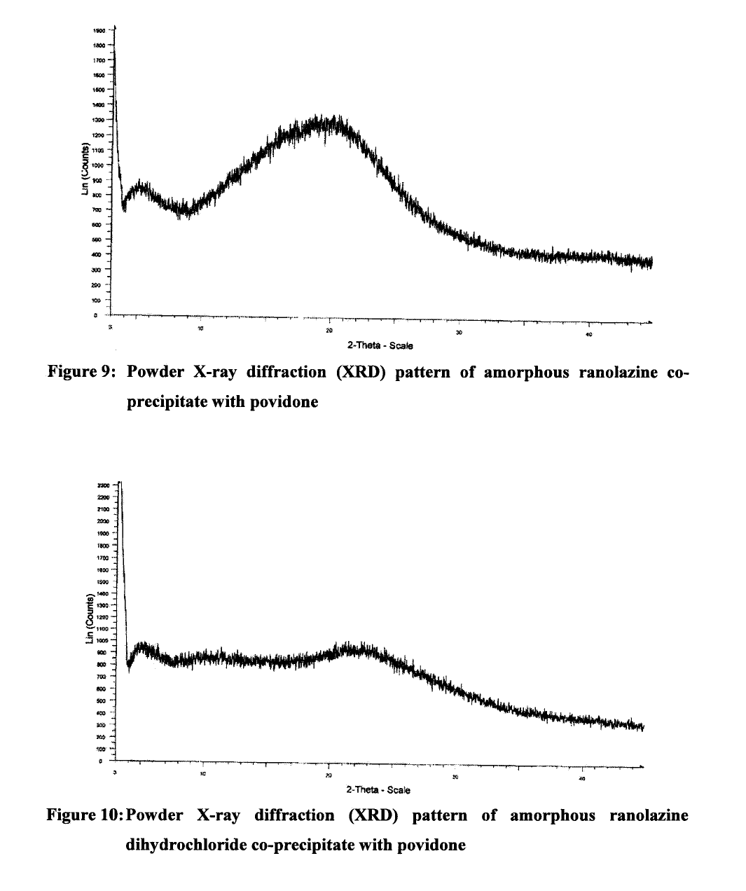

In one embodiment, the amorphous co-precipitate of ranolazine free base with povidone is characterized by a powder X-ray diffraction pattern substantially in accordance with FIG. 9.

In another embodiment, the amorphous co-precipitate of ranolazine dihydrochloride with povidone is characterized by a powder X-ray diffraction pattern substantially in accordance with FIG. 10.

According to another aspect, there is provided a process for the preparation of an amorphous co-precipitate of ranolazine or a pharmaceutically acceptable salt thereof with povidone, comprising:
a) providing a solution comprising ranolazine or a pharmaceutically acceptable salt thereof and povidone in a solvent;
b) optionally, filtering the solvent solution to remove any extraneous matter; and
c) substantially removing the solvent from the solution to produce the amorphous co-precipitate of ranolazine or a pharmaceutically acceptable salt thereof with povidone.

The process can produce amorphous co-precipitates of ranolazine or a pharmaceutically acceptable salt thereof with povidone in substantially pure form.

The amorphous co-precipitate comprising ranolazine or a pharmaceutically acceptable salt thereof and povidone obtained by the process disclosed herein is stable, consistently reproducible and has good flow properties, and which is particularly suitable for bulk preparation and handling, and so, the novel co-precipitates obtained by the process disclosed herein are suitable for formulating ranolazine or a pharmaceutically acceptable salt thereof.

Exemplary solvents used in step-(a) include, but are not limited to, water, an alcohol, a ketone, a chlorinated hydrocarbon, a nitrile, and mixtures thereof The term solvent also includes mixtures of solvents.

Specifically, the solvent is selected from the group consisting of water, methanol, ethanol, isopropyl alcohol, acetone, methylene chloride, acetonitrile, and mixtures thereof; and a most specific solvent is methanol.

The solution in step-(a) is provided either i) by dissolving ranolazine or a pharmaceutically acceptable salt thereof in the solvent and combining the solution with povidone, or ii) by dissolving ranolazine or a pharmaceutically acceptable salt thereof and povidone in the solvent.

The povidone may be used directly or in the form of a solution of povidone dissolved in the solvent selected from the group as described above.

In one embodiment, the ranolazine or a pharmaceutically acceptable salt thereof and povidone are dissolved in the solvent at a temperature of about 0° C. to about 140° C., specifically at about 20° C. to about 100° C., and more specifically at about 25° C. to about 80° C.

In another embodiment, the solution obtained in step-(a) is optionally stirred at a temperature of about 30° C. to the reflux temperature of the solvent used for at least 20 minutes, and specifically at a temperature of about 40° C. to about 100° C. for about 30 minutes to about 4 hours.

The solution obtained in step-(a) is optionally subjected to carbon treatment or silica gel treatment as per the methods described above.

The removal of the solvent from the solution in step-(c) is carried out by the methods as described above.

The amorphous co-precipitate comprising ranolazine or a pharmaceutically acceptable salt thereof and povidone obtained in step-(c) is recovered and further dried by the methods as described above.

In one embodiment, the dried product obtained by the process disclosed herein is optionally milled to get desired particle sizes. Milling or micronization can be performed prior to drying, or after the completion of drying of the product. The milling operation reduces the size of particles and increases surface area of particles. Drying is more efficient when the particle size of the material is smaller and the surface area is higher, hence milling is frequently performed prior to the drying operation.

Milling can be done suitably using jet milling equipment like an air jet mill, or using other conventional milling equipment.

The resulting amorphous powder compositions of the invention have improved solubility properties and hence also have improved bioavailability.

While the invention should not be constrained by any particular theory, the co-precipitates have the characteristics of solid dispersions at a molecular level, being in the nature of solid solutions. The solid solutions, or molecular dispersions, provide homogeneous particles in which no discrete areas of only amorphous ranolazine or a pharmaceutically acceptable salt thereof and only pharmaceutically acceptable excipient can be observed.

Further encompassed herein is the use of the solid state form of a ranolazine salt for the manufacture of a pharmaceutical composition together with a pharmaceutically acceptable carrier, wherein the salt of ranolazine is an oxalate salt, a maleate salt, a fumarate salt, a besylate salt, a tosylate salt or a dihydrobromide salt.

A specific pharmaceutical composition of the solid state form of ranolazine salt is selected from a solid dosage form and an oral suspension.

In one embodiment, the solid state form of ranolazine salt has a D₉₀ particle size of less than or equal to about 500 microns, specifically less than or equal to about 300 microns, more specifically less than or equal to about 100 microns, still more specifically less than or equal to about 60 microns, and most specifically less than or equal to about 15 microns, wherein the salt of ranolazine is an oxalate salt, a maleate salt, a fumarate salt, a besylate salt, a tosylate salt or a dihydrobromide salt.

In another embodiment, the amorphous co-precipitate of ranolazine or a pharmaceutically acceptable salt thereof with povidone has a D₉₀ particle size of less than or equal to about 500 microns, specifically less than or equal to about 300 microns, more specifically less than or equal to about 100 microns, still more specifically less than or equal to about 60 microns, and most specifically less than or equal to about 15 microns, wherein the pharmaceutically acceptable salt of ranolazine is a dihydrochloride salt, a dihydrobromide salt, an oxalate salt, a maleate salt, a fumarate salt, a besylate salt, a tosylate salt or a tartrate salt.

In another embodiment, the particle sizes of the solid state form of ranolazine salt, or the amorphous co-precipitate of ranolazine or a pharmaceutically acceptable salt thereof with povidone, are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided pharmaceutical compositions comprising the solid state form of ranolazine salt and one or more pharmaceutically acceptable excipients, wherein the salt of ranolazine is an oxalate salt, a maleate salt, a fumarate salt, a besylate salt, a tosylate salt or a dihydrobromide salt.

According to another aspect, there is provided pharmaceutical compositions comprising the solid state form of ranolazine salt prepared according to process disclosed herein and one or more pharmaceutically acceptable excipients, wherein the salt of ranolazine is an oxalate salt, a maleate salt, a fumarate salt, a besylate salt, a tosylate salt or a dihydrobromide salt.

According to another aspect, there is provided a process for preparing a pharmaceutical formulation comprising combining the solid state form of ranolazine salt prepared according to processes disclosed herein, with one or more pharmaceutically acceptable excipients, wherein the salt of ranolazine is an oxalate salt, a maleate salt, a fumarate salt, a besylate salt, a tosylate salt or a dihydrobromide salt.

According to another aspect, there is provided a method for treating a patient suffering from cardiovascular diseases including arrhythmias, variant and exercise induced angina and myocardial infarction; comprising administering a solid state form of ranolazine salt, or a pharmaceutical composition that comprises the solid state form of ranolazine salt along with pharmaceutically acceptable excipients, wherein the salt of ranolazine is an oxalate salt, a maleate salt, a fumarate salt, a besylate salt, a tosylate salt or a dihydrobromide salt.

According to another aspect, there is provided pharmaceutical composition comprising the amorphous form of ranolazine dihydrochloride having a water content of less than about 0.5% by weight based on the total weight of the amorphous ranolazine dihydrochloride and one or more pharmaceutically acceptable excipients.

According to another aspect, there is provided pharmaceutical compositions comprising the amorphous form of ranolazine dihydrochloride having a water content of less than about 0.5% by weight based on the total weight of the amorphous ranolazine dihydrochloride prepared according to process disclosed herein and one or more pharmaceutically acceptable excipients.

According to another aspect, there is provided a process for preparing a pharmaceutical formulation comprising combining the amorphous form of ranolazine dihydrochloride having a water content of less than about 0.5% by weight based on the total weight of the amorphous ranolazine dihydrochloride prepared according to processes disclosed herein, with one or more pharmaceutically acceptable excipients.

According to another aspect, there is provided pharmaceutical compositions comprising the amorphous co-precipitate of ranolazine or a pharmaceutically acceptable salt thereof with povidone and one or more pharmaceutically acceptable excipients, wherein the pharmaceutically acceptable salt of ranolazine is a dihydrochloride salt, a dihydrobromide salt, an oxalate salt, a maleate salt, a fumarate salt, a besylate salt, a tosylate salt or a tartrate salt.

According to another aspect, there is provided pharmaceutical compositions comprising the amorphous co-precipitate of ranolazine or a pharmaceutically acceptable salt thereof with povidone prepared according to process disclosed herein and one or more pharmaceutically acceptable excipients, wherein the pharmaceutically acceptable salt of ranolazine is a dihydrochloride salt, a dihydrobromide salt, an oxalate salt, a maleate salt, a fumarate salt, a besylate salt, a tosylate salt or a tartrate salt.

According to another aspect, there is provided a process for preparing a pharmaceutical formulation comprising combining the amorphous co-precipitate of ranolazine or a pharmaceutically acceptable salt thereof with povidone prepared according to processes disclosed herein, with one or more pharmaceutically acceptable excipients, wherein the pharmaceutically acceptable salt of ranolazine is a dihydrochloride salt, a dihydrobromide salt, an oxalate salt, a maleate salt, a fumarate salt, a besylate salt, a tosylate salt or a tartrate salt.

Yet in another embodiment, pharmaceutical compositions comprise at least a therapeutically effective amount of solid state form of a ranolazine salt, wherein the salt of ranolazine is an oxalate salt, a maleate salt, a fumarate salt, a besylate salt, a tosylate salt or a dihydrobromide salt. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The solid state form of ranolazine salt may also be administered as suppositories, ophthalmic ointments and suspensions, and parenteral suspensions, which are administered by other routes, wherein the salt of ranolazine is an oxalate salt, a maleate salt, a fumarate salt, a besylate salt, a tosylate salt or a dihydrobromide salt.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described herein.

In one embodiment, capsule dosage forms contain crystalline form of ranolazine salt within a capsule which may be coated with gelatin. Tablets and powders may also be coated with an enteric coating. Suitable enteric coating include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials such as powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### Instrumental Details:

### X-Ray Powder Diffraction (P-XRD):

The X-Ray powder diffraction was measured by an X-ray powder Diffractometer equipped with CuKα-radiations (40 kV, 40 mA) in wide-angle X-ray Diffractometer of BRUKER axs, D8 ADVANCE. The sample was analyzed using the following instrument parameters: measuring range=3-45 ° 2-theta; step width=0.01579°; and measuring time per step=0.11 sec.

The following examples are given for the purpose of illustrating the present disclosure and should not be considered as limitation on the scope or spirit of the disclosure.

### EXAMPLES

### Example 1

### Preparation of Crystalline Ranolazine Oxalate

Ranolazine base (2 g) was dissolved in isopropyl alcohol (14 ml) at 22-25° C., followed by slow addition of a solution of oxalic acid (1.3 g) in isopropyl alcohol (10 ml) at 22-25° C. The resulting mass was stirred for 4 hours at 22-25° C. The resulting solid was filtered, washed with isopropyl alcohol/hexane (5 ml/10 ml) and then dried under vacuum at 50-60° C. to yield 2.2 g of crystalline ranolazine oxalate (Melting Range: 142-148° C.).

### Example 2

### Preparation of Crystalline Ranolazine Maleate

Ranolazine base (2 g) was dissolved in isopropyl alcohol (14 ml) at 22-25° C., followed by slow addition of a solution of maleic acid (1.2 g) in isopropyl alcohol (10 ml) at 22-25° C. The resulting mass was stirred for 3 hours at 22-25° C. The separated solid was filtered and washed with isopropyl alcohol/hexane (5 ml/10 ml) and then dried under vacuum at 50-60° C. to yield 2.5 g of crystalline ranolazine maleate (Melting Range: 142-148° C.).

### Example 3

### Preparation of Crystalline Ranolazine Fumarate

Ranolazine base (2 g) was dissolved in isopropyl alcohol (14 ml) at 22-25° C., followed by slow addition of a solution of fumeric acid (1.2 g) in isopropyl alcohol (30 ml) at 22-25° C. The resulting mass was stirred for 4 hour at 22-25° C. The separated solid was filtered and washed with isopropyl alcohol (5 ml) and then dried under vacuum at 50-60° C. to yield 2.2 g of crystalline ranolazine fumarate (Melting Range: 109-150° C.).

### Example 4

### Preparation of Crystalline Ranolazine Besylate

Ranolazine base (2 g) was dissolved in isopropyl alcohol (100 ml) at 42-45° C., followed by the slow addition of benzenesulfonic acid (1.6 g) at 42-45° C. The reaction mixture was cooled to 0-5° C. and stirred for 2 hours. The separated solid was filtered and washed with isopropyl alcohol (5 ml) and then dried under vacuum at 50-60° C. to yield 2.3 g of crystalline ranolazine besylate.

### Example 5

### Preparation of Crystalline Ranolazine Tosylate

Ranolazine base (1 g) was dissolved in isopropyl alcohol (7 ml) at 22-25° C., followed by the slow addition of p-toluenesulfonic acid (1 g) at 22-25° C. The reaction mixture was cooled to 0-5° C. and stirred for 2 hours. The separated solid was filtered and washed with isopropyl alcohol (2 ml) and then dried under vacuum at 50-60° C. to yield 1.3 g of crystalline ranolazine tosylate (Melting Range: 162-168° C.).

### Example 6

Preparation of Amorphous Ranolazine dihydrobromide

Ranolazine base (2 g) was dissolved in methanol (5 ml) at 22-25° C., followed by the addition of 49% aqueous hydrobromic acid (0.5 ml) at 22-25° C. over a period of 10 minutes and stirring for 1 hour 30 minutes. The resulting solid was isolated by adding diethyl ether (50 ml) and then dried under vacuum at 50-60° C. to yield 1.2 g of amorphous ranolazine dihydrobromide.

### Example 7

Preparation of Crystalline Ranolazine dihydrobromide

Ranolazine base (5 g) was dissolved in methanol (25 ml) at 30-35° C., followed by the addition of 49% aqueous hydrobromic acid (3 ml) at 30-35° C. over a period of 10 minutes and stirring for 60 minutes. The resulting solid was isolated by adding diethyl ether (175 ml) at 20-25° C. and then dried under vacuum at 50-60° C. to yield 6.8 g of crystalline ranolazine dihydrobromide.

### Example 8

Preparation of Amorphous Ranolazine dihydrochloride

Ranolazine dihydrochloride (5 g) was dissolved in a mixture of methylene chloride (100 ml) and methanol (10 ml). The resulting solution was spray dried at 50° C. and the resulting solid was collected to yield 2.2 g of amorphous ranolazine dihydrochloride.

### Example 9

Preparation of Amorphous Coprecipitate of Ranolazine with Povidone

Ranolazine base (6 g) was dissolved in methanol (150 ml) at 25-27° C. Povidone (3 g) was added to the solution at 25-27° C. and then distilled off methanol completely under vacuum at 55° C., and the resulting residue was dried under vacuum at 60° C. followed by cooling to 25-30° C. to yield 6 g of amorphous coprecipitate of ranolazine with povidone.

### Example 10

Preparation of Amorphous Coprecipitate of Ranolazine Dihydrochloride with Povidone

Ranolazine dihydrochloride (1 g) was dissolved in methanol (50 ml) at 25-30° C. and followed by the addition of povidone (0.5 g). Methanol was distilled off from the solution under reduced pressure at 60° C. and the resulting residue was dried under vacuum at 60° C. followed by cooling to 25-30° C. to yield 1.2 g of amorphous coprecipitate of ranolazine dihydrochloride with povidone.

Unless otherwise indicated, the following definitions are set forth to illustrate and define the meaning and scope of the various terms used to describe the invention herein.

The term “solid state form of ranolazine salt disclosed herein” includes crystalline forms, amorphous form, hydrated, and solvated forms of ranolazine salt.

The term “crystalline form” refers to a crystal modification that can be characterized by analytical methods such as X-ray powder diffraction, IR-spectroscopy, differential scanning calorimetry (DSC) or by its melting point.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type) is another useful wetting agent, combinations thereof and other such materials known to those of ordinary skill in the art.

The term “micronization” used herein means a process or method by which the size of a population of particles is reduced.

As used herein, the term “micron” or “μm” refers to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent.

As used herein, D_{X} means that X percent of the particles have a diameter less than a specified diameter D. Thus, a D₉₀ or d(0.9) of less than 300 microns means that 90 volume-percent of the particles in a composition have a diameter less than 300 microns.

The term “coprecipitate or co-precipitate” as used herein refers to compositions comprising amorphous ranolazine or a pharmaceutically acceptable salt thereof together with povidone, being prepared by removing solvent from a solution containing both of them.

By “substantially pure” is meant having purity greater than about 98%, specifically greater than about 99%, and more specifically greater than about 99.9% measured by HPLC.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The term wt % refers to percent by weight. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Solid state form of a salt of 1-[3-(2-methoxyphenoxy-2-hydroxypropyl]-4-[(2,6-dimethylphenyl)aminocarbonylmethyl]piperazine (ranolazine salt), wherein the salt of ranolazine is an oxalate salt, a maleate salt, a fumarate salt, a besylate salt, a tosylate salt, or a dihydrobromide salt.

2. (canceled)

3. (canceled)

4. The solid state form of ranolazine salt of claim 1, having the following characteristics, wherein:
a) the solid state form of ranolazine oxalate salt is characterized by one or more of the following properties:
i) a powder X-ray diffraction pattern substantially in accordance with FIG. 1;
ii) a powder X-ray diffraction pattern having peaks at about 4.79, 16.58, 22.27 and 23.13±0.2 degrees 2-theta; and
iii) a powder X-ray diffraction pattern having additional peaks at about 9.65, 11.21, 12.60, 12.86, 13.25, 19.36, 22.79, 24.22 and 33.51±0.2 degrees 2-theta;
b) the solid state form of ranolazine maleate salt is characterized by one or more of the following properties:
i) a powder X-ray diffraction pattern substantially in accordance with FIG. 2;
ii) a powder X-ray diffraction pattern having peaks at about 6.25, 16.92, 24.82 and 25.54±0.2 degrees 2-theta; and
iii) a powder X-ray diffraction pattern having additional peaks at about 5.51, 8.78, 9.81, 11.12, 11.69, 12.69, 14.08, 15.82, 17.48, 18.55, 19.14, 19.68, 22.55, 26.68, 27.22, 28.64, 32.19 and 37.81±0.2 degrees 2-theta;
c) the solid state form of ranolazine fumarate salt is characterized by one or more of the following properties:
i) a powder X-ray diffraction pattern substantially in accordance with FIG. 3;
ii) a powder X-ray diffraction pattern having peaks at about 9.71, 10.53 and 19.27±0.2 degrees 2-theta; and
iii) a powder X-ray diffraction pattern having additional peaks at about 6.26, 9.04, 14.25, 15.17, 16.43, 16.62, 17.02, 18.15, 18.81, 19.51, 21.20, 21.66, 22.63, 23.41, 23.56, 24.01, 25.20, 25.81 and 27.91±0.2 degrees 2-theta;
d) the solid state form of ranolazine besylate salt is characterized by at least one, or more, of the following properties:
i) a powder X-ray diffraction pattern substantially in accordance with FIG. 4;
ii) a powder X-ray diffraction pattern having peaks at about 5.99, 17.64 and 19.04±0.2 degrees 2-theta; and
iii) a powder X-ray diffraction pattern having additional peaks at about 11.22, 12.01, 15.42, 16.77, 17.02, 21.86 and 23.65±0.2 degrees 2-theta;
e) the solid state form of ranolazine tosylate salt is characterized by at least one, or more, of the following properties:
i) a powder X-ray diffraction pattern substantially in accordance with FIG. 5;
ii) a powder X-ray diffraction pattern having peaks at about 5.74, 16.65, 18.05 and 18.76±0.2 degrees 2-theta; and
iii) a powder X-ray diffraction pattern having additional peaks at about 12.68, 15.35, 15.66, 16.94, 17.36, 21.63, 21.96 and 22.40±0.2 degrees 2-theta;
f) the solid state form of ranolazine dihydrobromide salt is characterized by at least one, or more, of the following properties:
i) a powder X-ray diffraction pattern substantially in accordance with FIG. 6; or
ii) a powder X-ray diffraction pattern substantially in accordance with FIG. 7;
iii) a powder X-ray diffraction pattern having peaks at about 5.31, 9.18, 15.86, 19.23 and 21.97±0.2 degrees 2-theta; and
iv) a powder X-ray diffraction pattern having additional peaks at about 10.25, 11.84, 16.92, 17.74, 18.27, 18.70, 19.96, 20.51, 21.49, 23.64, 24.21, 24.88, 25.47, 25.87, 26.57, 27.34, 27.63, 29.05, 29.68, 30.76, 32.64 and 34.20±0.2 degrees 2-theta.

5. A process for the preparation of solid state form of ranolazine salt of claim 1, comprising:
a) providing a first solution or a suspension of ranolazine free base in a solvent;
b) combining the first solution or suspension with an acid to produce a second solution or suspension containing a ranolazine acid addition salt, wherein the acid is selected from the group consisting of an oxalic acid, a maleic acid, a fumaric acid, a benzenesulfonic acid, a toluenesulfonic acid, and a hydrobromic acid; and
c) isolating and/or recovering the solid state form of ranolazine salt from the second solution or suspension obtained in step-(b), wherein the isolation of pure solid state form of ranolazine salt is carried out by forcible crystallization, spontaneous crystallization, substantial removal of the solvent from the solution or suspension, or a combination thereof.

6. (canceled)

7. The process of claim 65, wherein the solvent used in step-(a) is selected from the group consisting of water, methanol, ethanol, isopropyl alcohol, acetone, methylene chloride, and mixtures thereof; and wherein the acid in step-(b) is used in a molar ratio of about 2.0 to 2.5 moles per mole of ranolazine free base.

8. The process of claim 5, wherein the first solution in step-(a) is prepared by dissolving ranolazine free base in the solvent at a temperature of above about 20° C.; wherein the suspension in step-(a) is provided by suspending ranolazine free base in the solvent while stirring at a temperature of about 0° C. to the reflux temperature of the solvent used wherein the first solution or suspension obtained in step-(a) is optionally stirred at a temperature of about 25° C. to the reflux temperature of the solvent for about 15 minutes to about 8 hours; wherein the second solution obtained in step-(b) is optionally subjected to carbon treatment or silica gel treatment; wherein the crystallization in step-(c) is initiated by cooling, seeding, partial removal of the solvent from the solution, by adding an anti-solvent to the solution, or a combination thereof; wherein the removal of solvent in step-(c) is accomplished by substantially complete evaporation of the solvent, concentrating the solution or distillation of solvent under inert atmosphere, spray drying, vacuum drying, agitated thin-film (ATFD) drying, or a combination thereof; and wherein the recovering in step-(c) is carried out by filtration, filtration under vacuum, decantation, centrifugation, filtration employing a filtration media of a silica gel or celite, or a combination thereof.

9. The process of claim 8, wherein the ranolazine free base in step-(a) is dissolved in the solvent at a temperature of about 25° C. to about 100° C.; and wherein the suspension in step-(a) is stirred at a temperature of about 20° C. to about 100° C. for at least 30 minutes; wherein the anti-solvent is selected from the group consisting of n-pentane, n-hexane, n-heptane, cyclohexane, toluene, xylene, tetrahydrofuran, dioxane, diethyl ether, diisopropyl ether, monoglyme, diglyme, and mixtures thereof; and wherein the crystallization in step-(c) is carried out by cooling the solution while stirring at a temperature of about 0° C. to about 25° C.

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. A process for preparation of ranolazine or a dihydrochloride salt thereof, comprising converting a solid state form of a ranolazine salt to ranolazine or a dihydrochloride salt thereof, wherein the salt of ranolazine is an oxalate salt, a maleate salt, a fumarate salt, a besylate salt, a tosylate salt or a dihydrobromide salt.

27. (canceled)

28. The process of claim 26, wherein the solid state form of the ranolazine salt is the ranolazine dihydrobromide salt in an amorphous form, is prepared by a process comprising:
a) providing a solution of ranolazine free base in methanol in an amount of less than about 3.5 ml per gram of ranolazine free base;
b) combining the solution obtained in step-(a) with hydrobromic acid; and
c) isolating the amorphous form of ranolazine dihydrobromide by adding an anti-solvent, wherein the anti-solvent is diethyl ether or diisopropyl ether.

29. (canceled)

30. (canceled)

31. (canceled)

32. (canceled)

33. The process of claim 26, wherein the solid state form of the ranolazine salt is the ranolazine dihydrobromide salt in amorphous form, prepared by a process comprising:
a) providing a first solution of ranolazine free base in a solvent, wherein the solvent is selected from the group consisting of water, methanol, ethanol, isopropyl alcohol, acetone, methylene chloride, and mixtures thereof;
b) combining the first solution obtained in step-(a) with hydrobromic acid to produce a second solution; and
c) substantially removing the solvent from the second solution to produce the amorphous form of ranolazine dihydrobromide, wherein the removal of solvent is accomplished by substantially complete evaporation of the solvent, concentrating the solution or distillation of solvent under inert atmosphere, spray drying, vacuum drying, agitated thin-film (ATFD) drying, or a combination thereof.

34. (canceled)

35. (canceled)

36. (canceled)

37. The process of claim 265, wherein the solid state form of ranolazine salt is the ranolazine dihydrobromide salt in a crystalline form, prepared by a process comprising:
a) providing a solution of ranolazine free base in methanol in an amount of at least about 4.5 ml per gram of ranolazine free base;
b) combining the solution obtained in step-(a) with hydrobromic acid; and
c) isolating the crystalline ranolazine dihydrobromide salt by adding an anti-solvent, wherein the anti-solvent is diethyl ether or diisopropyl ether.

38. (canceled)

39. (canceled)

40. (canceled)

41. (canceled)

42. (canceled)

43. (canceled)

44. (canceled)

45. (canceled)

46. (canceled)

47. An amorphous co-precipitate comprising ranolazine or a pharmaceutically acceptable salt thereof and povidone (polyvinylpyrrolidone), wherein the pharmaceutically acceptable salt of ranolazine is a dihydrochloride salt, a dihydrobromide salt, an oxalate salt, a maleate salt, a fumarate salt, a besylate salt, a tosylate salt or a tartrate salt.

48. (canceled)

49. The amorphous co-precipitate of claim 47, wherein the amorphous co-precipitate of ranolazine free base with povidone is characterized by a powder X-ray diffraction pattern substantially in accordance with FIG. 9; and wherein the amorphous co-precipitate of ranolazine dihydrochloride with povidone is characterized by a powder X-ray diffraction pattern substantially in accordance with FIG. 10.

50. (canceled)

51. A process for the preparation of an amorphous co-precipitate of ranolazine or a pharmaceutically acceptable salt thereof with povidone of claim 47, comprising:
a) providing a solution comprising ranolazine or a pharmaceutically acceptable salt thereof and povidone in a solvent, wherein the solvent is selected from the group consisting of water, methanol, ethanol, isopropyl alcohol, acetone, methylene chloride, acetonitrile, and mixtures thereof;
b) optionally, filtering the solvent solution; and
c) substantially removing the solvent from the solution to produce the amorphous co-precipitate of ranolazine or a pharmaceutically acceptable salt thereof with povidone, wherein the removal of solvent is accomplished by substantially complete evaporation of the solvent concentrating the solution or distillation of solvent under inert atmosphere, spray drying, vacuum drying, agitated thin-film (ATFD) drying, or a combination thereof.

52. (canceled)

53. (canceled)

54. (canceled)

55. (canceled)

56. (canceled)

57. The solid state form of a ranolazine salt of claim 1, further comprising one or more pharmaceutically acceptable excipients to form a pharmaceutical composition.

58. (canceled)

59. The amorphous co-precipitate of ranolazine or a pharmaceutically acceptable salt thereof with povidone of claim 47, further comprising one or more pharmaceutically acceptable excipients to form a pharmaceutical composition.

60. (canceled)

61. The pharmaceutical composition of claim 57, wherein the solid state form of ranolazine salt has a D₉₀ particle size of less than or equal to about 500 microns.

62. (canceled)

63. The pharmaceutical composition of claim 61, wherein the D₉₀ particle size is less than or equal to about 300 microns; less than or equal to about 100 microns; less than or equal to about 60 microns; or less than or equal to about 15 microns.

64. (canceled)
